(19) United States
(12) Patent Application Publication
(10) Pub. No.: US 2021/0252042 A1
(43) Pub. Date: Aug. 19, 2021

(54) METHOD FOR SCAVENGING HYDROGEN PEROXIDE AND REGULATING GASTROINTESTINAL FUNCTION BY USING DRINKING WATER COMPRISING SILICON DIOXIDE

(71) Applicant: PROFIT SUCCESS INC., Taoyuan City (TW)

(72) Inventor: Ming-Chih Chen, Taoyuan City (TW)

(21) Appl. No.: 16/922,411

(22) Filed: Jul. 7, 2020

(30) Foreign Application Priority Data
Feb. 14, 2020 (TW) 109201646

(51) Int. Cl. A61K 33/00; A61K 9/00; C02F 1/42; C02F 1/48; A23L 2/52; A23L 33/16

(52) U.S. Cl. CPC A61K 33/00; A61K 9/0095; C02F 1/42; C02F 2307/10; A23L 2/52; A23L 33/16; C02F 1/48

(57) ABSTRACT

The present disclosure provides a method for scavenging hydrogen peroxide and regulating gastrointestinal function by using a drinking water including silicon dioxide (SiO₂).

### CROSS-REFERENCE TO RELATED APPLICATION

This application claims priority of Taiwan patent application No. 109201646, filed on Feb. 14, 2020, the content of which is incorporated herein in its entirety by reference.

### BACKGROUND OF THE INVENTION

### 1. Field of the Invention

The present invention relates to a method for scavenging hydrogen peroxide and regulating gastrointestinal function by using a drinking water comprising silicon dioxide (SiO₂).

### 2. The Prior Art

Mounting evidence indicates the important role that silicon can play in health. However, the mechanisms of action of silicon remain unclear. Studies have demonstrated the antioxidant and antiapoptotic properties of organic silicon in a human neuroblastoma cell line. In addition, the beneficial effect of silicon incorporated in a restructured pork (RP) matrix has been analyzed in aged rats fed a high-saturated fat, high-cholesterol diet (HSHCD). Moreover, dietary enrichment with silicon enhanced hepatocyte antioxidant defenses, apparently by removing hydrogen peroxide.

However, the availability of silicon is not easy, and it is more difficult to commercially prepare food products or drinking water comprising silicon for large-scale production. Therefore, those skilled in the art urgently need to develop a setup for producing drinking water comprising silicon dioxide (SiO₂) and to determine its effect on SiO₂ production and reducing reactive oxygen species. The inventors also evaluate the effect of the drinking water on the gastrointestinal function, gut microbiota and immune modulation by oral administration of the drinking water.

### SUMMARY OF THE INVENTION

A primary objective of the present invention is to provide a method for scavenging hydrogen peroxide and beneficially regulating gastrointestinal function, comprising administering to a subject in need thereof a drinking water comprising an effective amount of silicon dioxide (SiO₂).

According to an embodiment of the present invention, the beneficial regulation of gastrointestinal function comprises controlling body weight.

According to an embodiment of the present invention, the beneficial regulation of gastrointestinal function comprises regulating gastrointestinal motility.

According to an embodiment of the present invention, the beneficial regulation of gastrointestinal function comprises inhibiting gastric juice secretion.

According to an embodiment of the present invention, the beneficial regulation of gastrointestinal function comprises increasing gut microbiota.

According to an embodiment of the present invention, the beneficial regulation of gastrointestinal function comprises scavenging free radicals of stomach mucosa.

According to an embodiment of the present invention, the silicon dioxide (SiO₂) is in an amount of at least 0.1 mg/L.

According to an embodiment of the present invention, the drinking water is prepared by a drinking fountain.

According to an embodiment of the present invention, the drinking fountain comprises: an activated carbon column; at least one ion exchange resin column disposed adjacent to the activated carbon column; an activated filter column disposed adjacent to the at least one ion exchange resin column; a silicon minerals column disposed adjacent to the activated filter column; a UV sterilizer disposed adjacent to the silicon minerals column; and a magnetizer disposed adjacent to the UV sterilizer; wherein the silicon minerals column comprises silicon minerals, and the silicon minerals are prepared by stirring, mixing and sintering at a predetermined temperature, such that the silicon minerals are sintered into a crystalloid.

According to an embodiment of the present invention, the predetermined temperature ranges from 0° C. to 60° C.

According to an embodiment of the present invention, the silicon minerals form a mineral sphere having a diameter of 8 mm to 15 mm.

According to an embodiment of the present invention, the activated carbon column, the at least one ion exchange resin column, the activated filter column, the Si minerals column, the UV sterilizer, and the magnetizer communicate with each other, and the drinking water is obtained by passing a tap water through the activated carbon column, the at least one ion exchange resin column, the activated filter column, the Si minerals column, the UV sterilizer, and the magnetizer.

According to an embodiment of the present invention, the tap water sequentially passes through the activated carbon column, the at least one ion exchange resin column, the activated filter column, the Si minerals column, the UV sterilizer, and the magnetizer through a pressure gradient.

According to an embodiment of the present invention, the activated carbon column, the activated filter column, and the magnetizer are in cylindrical forms, respectively.

In summary, the present invention has the effect on scavenging hydrogen peroxide and regulating gastrointestinal function via controlling body weight, regulating gastrointestinal motility, inhibiting gastric juice secretion, increasing gut microbiota, and scavenging free radicals of stomach mucosa by using the drinking water comprising silicon dioxide (SiO₂).

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 is a schematic diagram of a drinking fountain for preparing drinking water comprising silicon dioxide (SiO₂).

FIG. 2 is another schematic diagram of the drinking fountain for preparing drinking water comprising silicon dioxide (SiO₂).

FIG. 3A is a photograph showing the Si minerals form a mineral sphere of 10 mm.

FIG. 3B is another photograph showing the Si minerals form a mineral sphere of 10 mm.

FIG. 4 shows a comparison of silicon dioxide concentrations in drinking water comprising silicon dioxide at different concentrations, wherein 0.5BT, 1BT, 2BT and 5BT represent drinking water comprising silicon dioxide diluted with 0.5 fold, 1 fold, 2 fold and 5 fold, respectively; * indicates p<0.05 compared with tap water.

FIG. 5 shows the effect of the drinking water comprising silicon dioxide on scavenging hydrogen peroxide, wherein * indicates p<0.05 compared with the comparative group.

FIG. 6A is a data diagram showing the effect of the drinking water comprising silicon dioxide on controlling body weight.

FIG. 6B is a data diagram showing the effect of the drinking water comprising silicon dioxide on controlling dry weight of feces, wherein * indicates p<0.05 compared with the control group.

FIG. 6C is a data diagram showing the effect of the drinking water comprising silicon dioxide on controlling dry weight of feces/body weight, wherein * indicates p<0.05 compared with the control group.

FIG. 7A is a photograph showing the effect of the drinking water comprising silicon dioxide on regulating gastrointestinal motility.

FIG. 7B is a data diagram showing the effect of the drinking water comprising silicon dioxide on regulating gastrointestinal motility, wherein * indicates p<0.05 compared with the control group.

FIG. 8A is a data diagram showing the effect of the drinking water comprising silicon dioxide on inhibiting gastric juice secretion, wherein * indicates p<0.05 compared with the control group.

FIG. 8B is a data diagram showing the effect of the drinking water comprising silicon dioxide on scavenging free radicals of stomach mucosa, wherein * indicates p<0.05 compared with the pathological control group; # indicates p<0.05 compared with the normal control group.

FIG. 8C is a histochemistry image showing the effect of the drinking water comprising silicon dioxide on scavenging free radicals of stomach mucosa.

FIG. 9 is a data diagram showing the effect of the drinking water comprising silicon dioxide on increasing gut microbiota.

FIG. 10A is a data diagrams showing the effect of the drinking water comprising silicon dioxide on increasing gut microbiota (Bifidobacterium).

FIG. 10B is a data diagrams showing the effect of the drinking water comprising silicon dioxide on increasing gut microbiota (Clostridium), wherein * indicates p<0.05.

FIG. 10C is a data diagrams showing the effect of the drinking water comprising silicon dioxide on increasing gut microbiota (Lactobacillus), wherein * indicates p<0.05.

FIG. 10D is a data diagrams showing the effect of the drinking water comprising silicon dioxide on increasing gut microbiota (Lactobacillus reuteri).

FIG. 10E is a data diagrams showing the effect of the drinking water comprising silicon dioxide on increasing gut microbiota (Lactobacillus murinus), wherein * indicates p<0.05.

FIG. 10F is a data diagrams showing the effect of the drinking water comprising silicon dioxide on increasing gut microbiota (Lactococcus), wherein * indicates p<0.05.

FIG. 10G is a data diagrams showing the effect of the drinking water comprising silicon dioxide on increasing gut microbiota (Weissella), wherein * indicates p<0.05.

FIG. 10H is a data diagrams showing the effect of the drinking water comprising silicon dioxide on increasing gut microbiota (Streptococcus), wherein * indicates p<0.05.

FIG. 10I is a data diagrams showing the effect of the drinking water comprising silicon dioxide on increasing gut microbiota (Bifidobacterium longum).

FIG. 10J is a data diagrams showing the effect of the drinking water comprising silicon dioxide on increasing gut microbiota (Escherichia shigella), wherein * indicates p<0.05.

FIG. 10K is a data diagrams showing the effect of the drinking water comprising silicon dioxide on increasing gut microbiota (Erysipelatoclostridium), wherein * indicates p<0.05.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

SigmaPlot 10.0 (Systat Software, Inc., Chicago, Ill., USA) software was used for graphing and statistical analysis. All experimental values were expressed as the mean±standard deviation. All parameters were compared by using Two-way analysis of variance (ANOVA) to assess the differences among groups. The value of P <0.05 was indicated as statistical significance.

### Example 1

### Setup of Drinking Fountain for Preparing Drinking Water Comprising Silicon Dioxide (SiO₂)

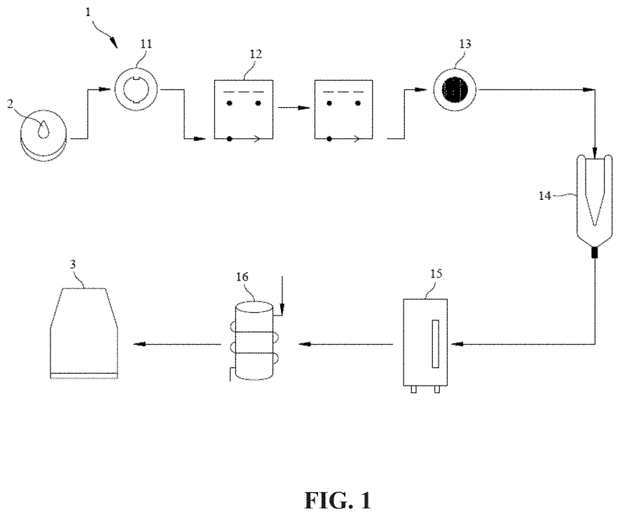

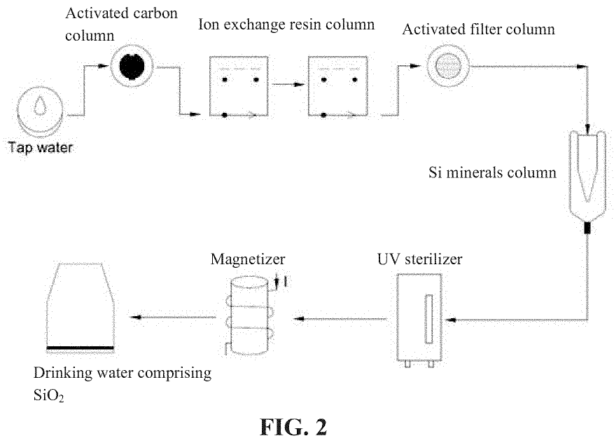

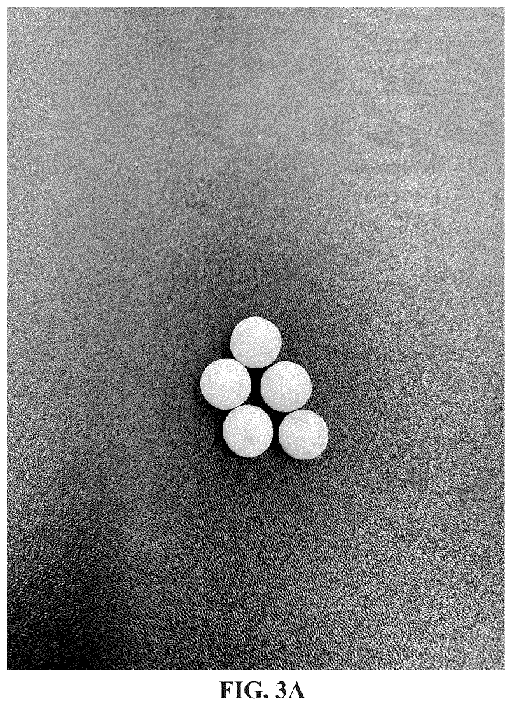

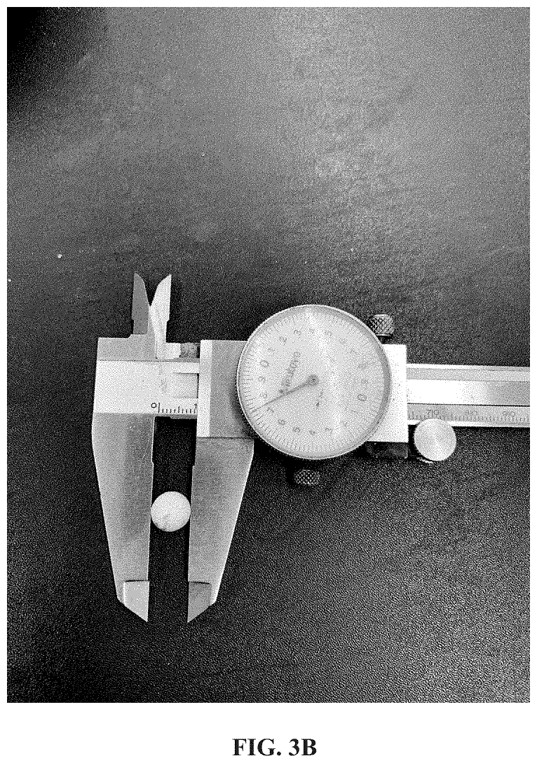

Referring to FIG. 1 and FIG. 2, which are schematic diagrams of a drinking fountain 1 for preparing drinking water comprising silicon dioxide (SiO₂). The drinking fountain 1 for preparing drinking water comprising silicon dioxide (SiO₂) comprises: an activated carbon column 11; at least one ion exchange resin column 12 disposed adjacent to the activated carbon column 11; an activated filter column 13 disposed adjacent to the at least one ion exchange resin column 12; a Si minerals column 14 disposed adjacent to the activated filter column 13; a UV sterilizer 15 disposed adjacent to the Si minerals column 14; and a magnetizer 16 disposed adjacent to the UV sterilizer 15; wherein the silicon minerals column 14 comprises silicon minerals, and the silicon minerals are prepared by stirring, mixing and sintering at 0° C. to 60° C., such that the silicon minerals are sintered into a crystalloid (see FIG. 3A and FIG. 3B). The content of silicon is up to 96.095%. Preferably, the silicon minerals column 14 comprises silicon minerals, and the silicon minerals are prepared by stirring, mixing and sintering at 800° C. The above mentioned columns were obtained from EARTHWATERFIRE LTD.

In this example, the silicon dioxide (SiO₂) is in an amount of at least 0.1 mg/L.

In this example, the silicon minerals form a mineral sphere having a diameter of 8 mm to 15 mm (see FIG. 3A and FIG. 3B). Preferably, the silicon minerals form a mineral sphere having a diameter of 10 mm.

In this example, the activated carbon column 11, the at least one ion exchange resin column 12, the activated filter column 13, the Si minerals column 14, the UV sterilizer 15, and the magnetizer 16 communicate with each other, and the drinking water 3 is obtained by passing a tap water 2 through the activated carbon column 11, the at least one ion exchange resin column 12, the activated filter column 13, the Si minerals column 14, the UV sterilizer 15, and the magnetizer 16.

In this example, the number of ion exchange resin columns 12 is preferably two columns.

In this example, the tap water 2 sequentially passes through the activated carbon column 11, the at least one ion exchange resin column 12, the activated filter column 13, the Si minerals column 14, the UV sterilizer 15, and the magnetizer 16 through a pressure gradient of 3.5 kg/cm² and is collected by a 5-ton chamber.

### Example 2

### Evaluation of the Effect of Drinking Water Comprising Silicon Dioxide on Scavenging Hydrogen Peroxide

The experimental animals used in the following experiments were forty male 7-week-old Wistar rats and fifty male 7-week-old C57BL/6 mice purchased from BioLASCO Taiwan Co., Ltd, (Yi-Lan, Taiwan). Animals were housed in the animal center of National Taiwan Normal University at a controlled room temperature under a 12 hours dark-light cycle with free access to food and tap water. After one week period of accommodation, rats and mice were divided in to six groups respectively, eight rats per group and ten mice per group, including a control group (tap water), a comparative group (RO distilled water treatment), Experimental group 1 (treated with drinking water comprising 0.5 mg/L silicon dioxide (after 12 hours of reflux through the Si minerals column 14, the UV sterilizer 15, and the magnetizer 16), Experimental group 2 (treated with drinking water comprising 1 mg/L silicon dioxide (after 24 hours of reflux)), Experimental group 3 (treated with drinking water comprising 2 mg/L silicon dioxide (after 48 hours of reflux)), and Experimental group 4 (treated with drinking water comprising 5 mg/L silicon dioxide).

All treatments were added in the drinking water for 4 weeks. Animal body weights were measured before and after treatments weekly. All experiments were performed in accordance with the guidelines of the National Science Council of the Republic of China (1997) and were approved by the Institutional Animal Care and Use Committee of National Taiwan Normal University (No. 107015).

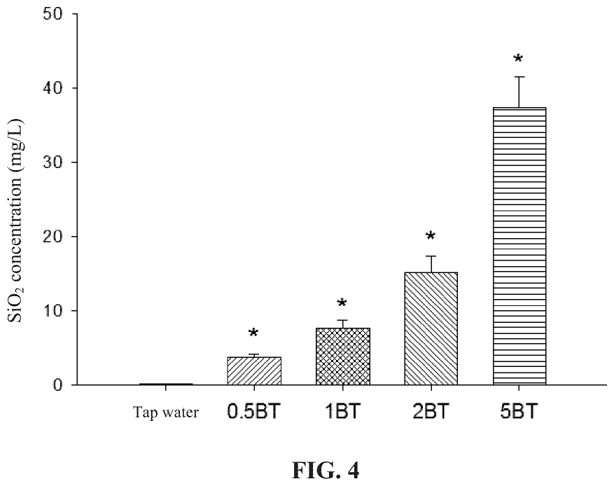

FIG. 4 shows a comparison of silicon dioxide concentrations in drinking water comprising silicon dioxide at different concentrations. As shown in FIG. 4, compared with tap water, the concentration of silicon dioxide in drinking water comprising silicon dioxide has increased significantly, and it becomes more obvious as the concentration of drinking water comprising silicon dioxide increases.

Subsequently, in vitro chemiluminescence recording for free radical was performed. The free radical level of the stomach tissue was measured by luminol chemiluminescence detection method. Concisely, a piece of freshly harvested stomach tissue was mixed with 0.5 mL of 0.1 mmol/L luminol (5-amino-2,3-dihydro-1,4-phthalazinedione, Sigma, Chemical Co., St. Louis, Mo.) and was analyzed with a chemiluminescence analyzing system (CLD-110, Tohoku Electronic Inc. Co., Sendai, Japan). The chemiluminescence signals emitted from the mix of stomach tissue and luminol, which represented the hydrogen peroxide content in the stomach lumen, were recorded for 240 sec. In addition, the inventors evaluated the free radical scavenging activity of all doses of drinking water comprising silicon dioxide. Briefly, 0.2 mL of test samples were mixed with 0.5 mL of luminol and 0.1 mL of H₂O₂ (0.03%) sequentially. The enhanced chemiluminescent signals form the sample-luminol-H₂O₂ mixture were recorded for 180 seconds. The total chemiluminescent (CL) counts, representing the hydrogen peroxide count in luminol detection method, were calculated from the area under the curve.

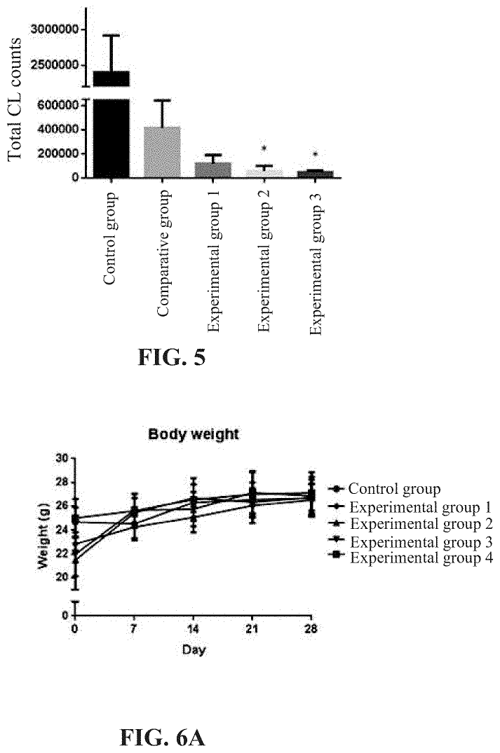

FIG. 5 and Table 1 show the effect of the drinking water comprising silicon dioxide on scavenging hydrogen peroxide. As shown in FIG. 5 and Table 1, compared with the control group and the comparative group, the total chemiluminescent (CL) counts of Experimental group 1 to Experimental group 3 were significantly decreased, and tend to be obvious as the concentration of drinking water comprising silicon dioxide increases. The result of this example indicates that the drinking water comprising silicon dioxide has the effect on scavenging hydrogen peroxide.

TABLE 1
Total chemiluminescent (CL) counts

| | |
|---|---|
| Control group | 2398554.6 ± 424006.4 |
| Comparative group | 416237.3 ± 185678.4 |
| Experimental group 1 | 120026 ± 56777 |
| Experimental group 2 | 59130.6 ± 34499.1 |
| Experimental group 3 | 49761.6 ± 10641.2 |

### Example 3

### Evaluation of the Effect of Drinking Water Comprising Silicon Dioxide on Controlling Body Weight and Regulating Gastrointestinal Motility

The experimental animals and grouping manners in this example are the same as those described in Example 2. After four weeks of experimental treatments, mice were housed in cage individually with free access to food and water. Fecal samples of mice were collected in a period of 24-hours for the measurement of daily dry weight of feces. Afterwards, all mice were fasted for 24 hours but with free access to water before experimentation. Gastrointestinal transit of mice were evaluated by the transport of a test meal containing non-absorbable marker, charcoal. In brief, the test meal (0.1 ml) was administered intragastrically by oral gavage feeding tube.

Thirty minutes after test meal administration, the mice were anesthetized with intraperitoneal injection of urethane (1.2 g/kg, Sigma-Aldrich, St. Louis, USA). After the small intestines of mice were harvested rapidly by laparotomy, mice were sacrificed by intravenous injection of potassium chloride. Gastrointestinal transit was shown as the percentage of the length of the small intestine traversed with the charcoal marker divided by the total length of the small intestine.

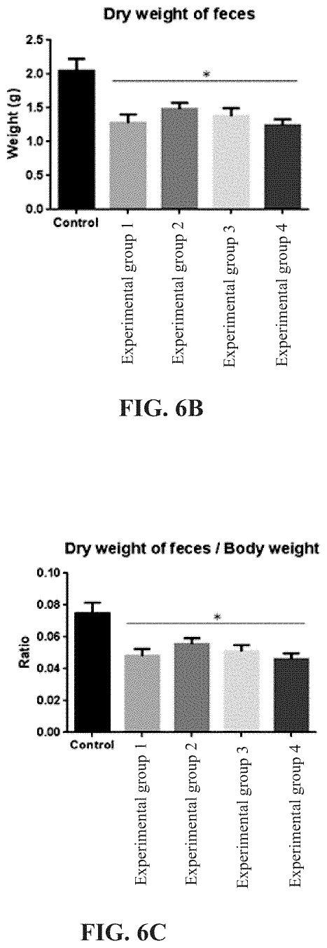

FIGS. 6A to 6C are data diagrams showing the effect of the drinking water comprising silicon dioxide on controlling body weight, dry weight of feces, and dry weight of feces/body weight. As shown in FIG. 6A, drinking water comprising silicon dioxide does not affect weight change and has no side effect. As shown in FIG. 6B, compared with the control group, the dry weight of feces of Experimental groups 1 to 4 was significantly reduced. The result of this experiment indicates that the drinking water comprising silicon dioxide has the effect on controlling body weight.

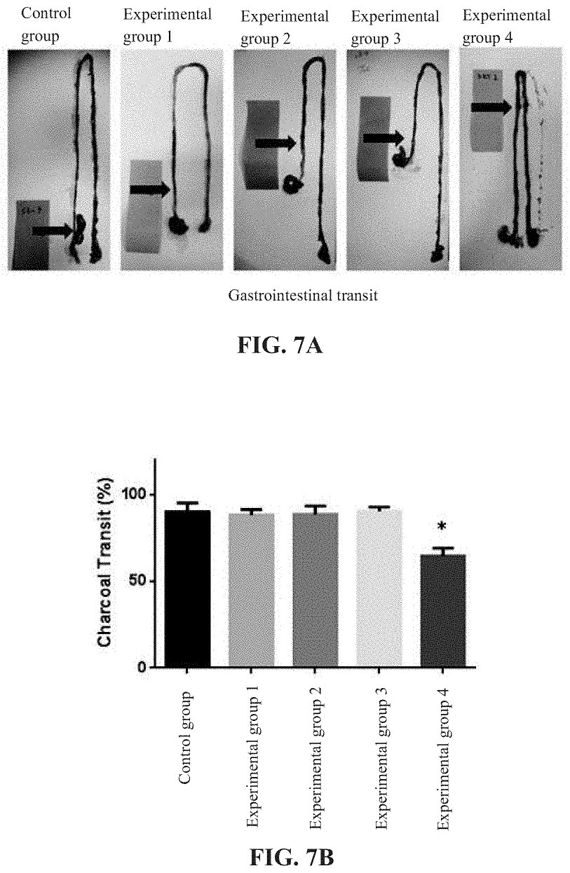

FIG. 7A is a photograph showing the effect of the drinking water comprising silicon dioxide on regulating gastrointestinal motility. FIG. 7B is a data diagram showing the effect of the drinking water comprising silicon dioxide on regulating gastrointestinal motility. As shown in FIGS. 7A and 7B, compared with the control group, the charcoal transit (%) of Experimental group 4 was significantly reduced.

The result of this experiment indicates that the drinking water comprising silicon dioxide has the effect on controlling body weight and regulating gastrointestinal motility, thereby achieving gastrointestinal regulation effect.

### Example 4

### Evaluation of the Effect of Drinking Water Comprising Silicon Dioxide on Inhibiting Gastric Juice Secretion and Scavenging Free Radicals of Stomach Mucosa

In this example, the experimental animals and the grouping manners for the experiment of inhibiting gastric juice secretion are the same as those described in Example 2. The experimental animals, grouping manners and experimental method of the free radicals of stomach mucosa scavenging experiment are substantially the same as those described in Example 2, except that the normal control group and the pathological control group are used instead of the control group, wherein the experimental animals in the normal control group were treated with tap water, and the experimental animals in the pathological control group were treated with pyloric ligation and tap water. After four weeks of experimental treatments, rats were fasted for 24 hours but with free access to water before experimentation. After the rat was anesthetized with urethane (I.P.), the stomach was exposed by midline laparotomy, and the pylorus ligation was conducted (the normal control group was not subjected to this treatment). Four hours after the pylorus ligation surgery, the stomach was harvested for histological observation, and the gastric juice was collected into graduated test tube to evaluate the effect of experimental treatments on the gastric secretion.

The stomach tissue were fixed with 10% formalin in phosphate-buffered saline for 24-hours and embedded in paraffin. Sections (5 μm) of stomach were sliced with microtome (RM 2125 RTS, LEICA, Germany) Sections were mounted on slides, and stained with hematoxylin and eosin (H&E) for pathological examinations. The inventors utilized light microscopic evaluation to analyze the histopathological changes of the stomach including leukocytes infiltration, erythrocytes extravasation and the damaged epithelial integrity of stomach mucosa.

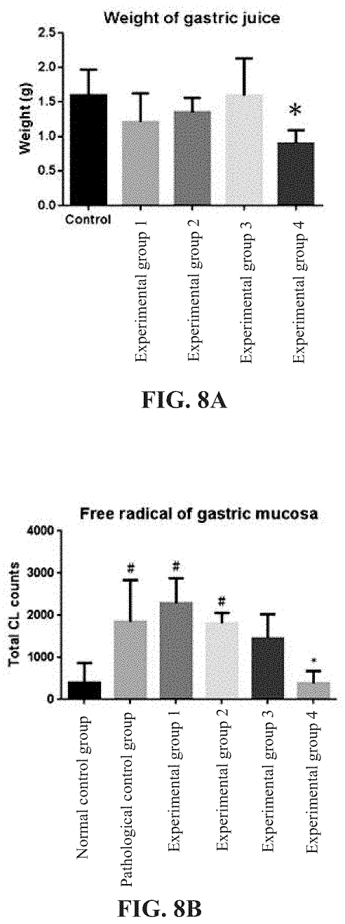

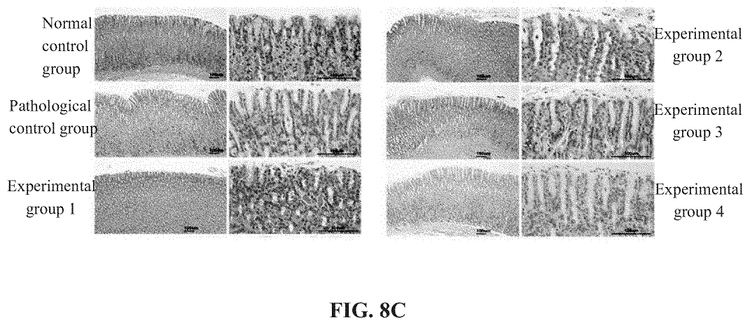

FIG. 8A is a data diagram showing the effect of the drinking water comprising silicon dioxide on inhibiting gastric juice secretion. As shown in FIG. 8A, compared with the control group, the weight of gastric juice of Experimental group 4 was significantly reduced. The result of this experiment indicates that the drinking water comprising silicon dioxide has the effect on inhibiting gastric juice secretion.

FIG. 8B is a data diagram showing the effect of the drinking water comprising silicon dioxide on scavenging free radicals of stomach mucosa. FIG. 8C is a histochemistry image showing the effect of the drinking water comprising silicon dioxide on scavenging free radicals of stomach mucosa. As shown in FIGS. 8B and 8C, compared with the normal control group, the total chemiluminescent (CL) counts of the pathological control group were significantly increased. Compared with the pathological control group, the total chemiluminescent (CL) counts of Experimental group 3 and Experimental group 4 were significantly reduced. The result of this experiment indicates that the drinking water comprising silicon dioxide has the effect on scavenging free radicals of stomach mucosa.

### Example 5

### Evaluation of the Effect of Drinking Water Comprising Silicon Dioxide on Increasing Gut Microbiota

In this example, the experimental animals and the grouping manners are the same as those described in Example 2. Fecal samples were collected from rats after 4 weeks of experimental treatments. Fecal microbiota genome was extracted using the QIAamp DNA Stool Mini Kit (Qiagen, USA). The next-generation sequencing of bacterial 16 S ribosomal RNA genes were conducted to distinguish the intestinal bacteria. The V3-V4 regions of 16S rRNA genes, which were generally used for intestinal microbiome studies, were amplified using a specific primer with a barcode. Fecal microbiota composition was assessed using Illumina HiSeq sequencing of 16S rDNA amplicon and QIIME-based microbiota analysis. Operational taxonomic unit (OTU) clustering and species annotation were performed from representative sequences using UPARSE software (Version 7.0.1001) and the Greengenes Database based on Ribosomal Database Project classifier (Version 2.2), respectively. OTUs abundance information was normalized with a standard of sequence number corresponding to the sample with the least sequences. OTU is annotated and divided into phylum, class, order, family, genus and species. The zero-inflated Gaussian mixture (ZIG) model of metagenomeSeq was used for the comparison of microbiota composition among groups. The microorganisms were identified via 16S RNA sequence analysis and the above statistical method.

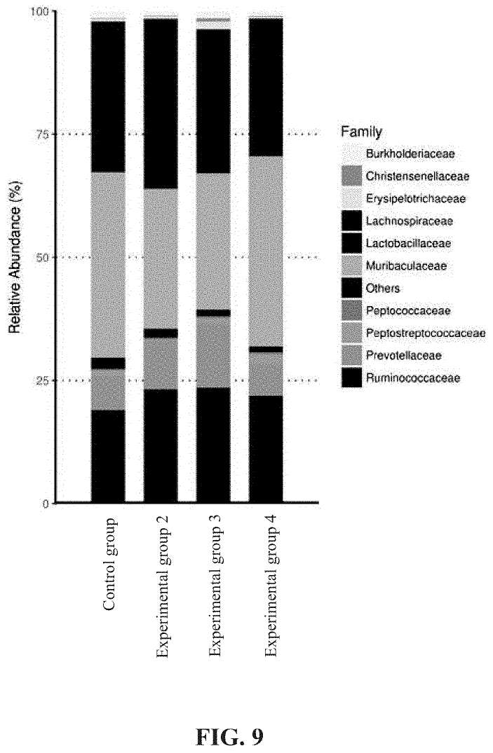

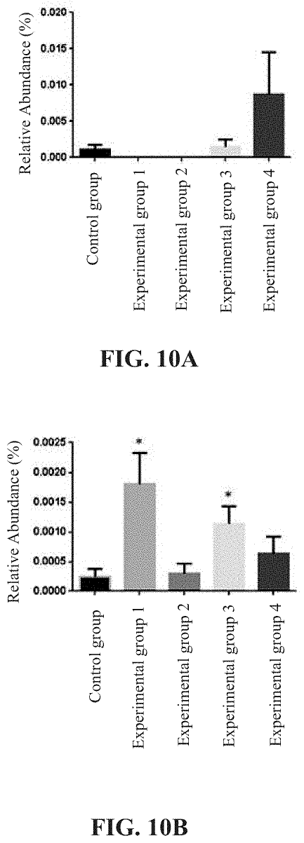

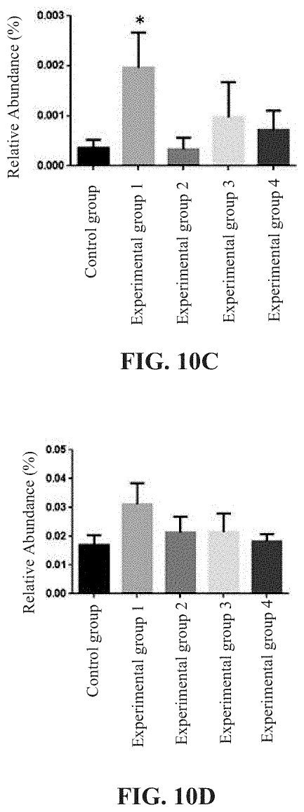

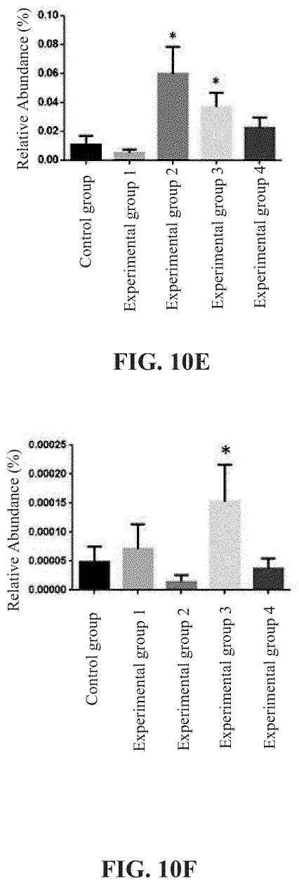

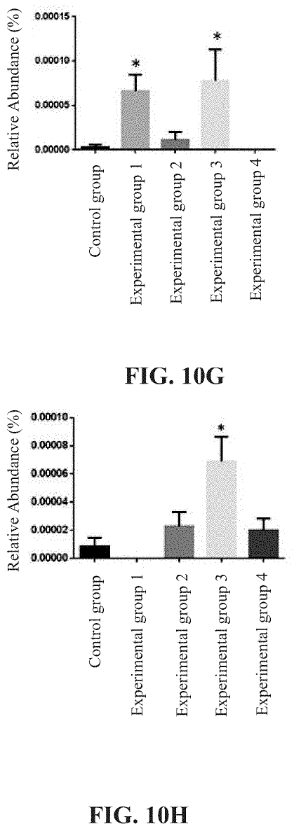

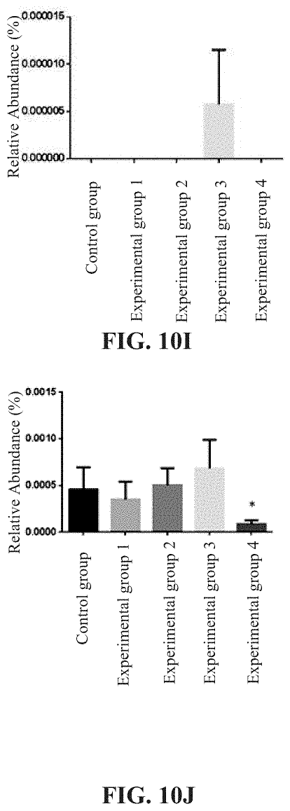

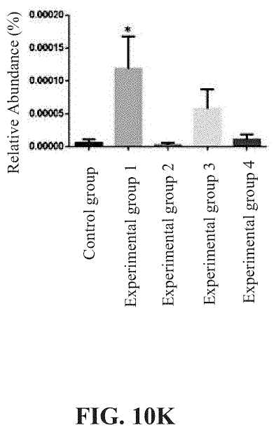

FIG. 9 and FIGS. 10A to 10K are data diagrams showing the effect of the drinking water comprising silicon dioxide on increasing gut microbiota. As shown in FIG. 9 and FIGS. 10A to 10K, compared with the control group, the relative abundances of gut microbiota in Experimental groups 1 to 4 (including Burkholderiaceae, Christensenellaceae, Erysipelotrichaceae, Lachnospiraceae, Lactobacillaceae, Muribaculaceae, Peptococcaceae, Peptostreptococcaceae, Prevotellaceae, Ruminococcaceae, Bifidobacterium, Clostridium, Lactobacillus, Lactobacillus reuteri, Lactobacillus murinus, Lactococcus, Weissella, Streptococcus, Bifidobacterium longum, Escherichia shigella, and Erysipelatoclostridium) were significantly increased. The result of this example indicates that the drinking water comprising silicon dioxide has the effect on increasing gut microbiota.

In summary, the present invention has the effect on scavenging hydrogen peroxide and regulating gastrointestinal function via controlling body weight, regulating gastrointestinal motility, inhibiting gastric juice secretion, increasing gut microbiota, and scavenging free radicals of stomach mucosa by using the drinking water comprising silicon dioxide (SiO₂).

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for scavenging hydrogen peroxide and beneficially regulating gastrointestinal function, comprising administering to a subject in need thereof a drinking water comprising an effective amount of silicon dioxide (SiO₂).

2. The method according to claim 1, wherein the beneficial regulation of gastrointestinal function comprises controlling body weight.

3. The method according to claim 1, wherein the beneficial regulation of gastrointestinal function comprises regulating gastrointestinal motility.

4. The method according to claim 1, wherein the beneficial regulation of gastrointestinal function comprises inhibiting gastric juice secretion.

5. The method according to claim 1, wherein the beneficial regulation of gastrointestinal function comprises increasing gut microbiota.

6. The method according to claim 1, wherein the beneficial regulation of gastrointestinal function comprises scavenging free radicals of stomach mucosa.

7. The method according to claim 1, wherein the silicon dioxide (SiO₂) is in an amount of at least 0.1 mg/L.

8. The method according to claim 1, wherein the drinking water is prepared by a drinking fountain.

9. The method according to claim 8, wherein the drinking fountain comprises:
an activated carbon column;
at least one ion exchange resin column disposed adjacent to the activated carbon column;
an activated filter column disposed adjacent to the at least one ion exchange resin column;
a silicon minerals column disposed adjacent to the activated filter column;
a UV sterilizer disposed adjacent to the silicon minerals column; and
a magnetizer disposed adjacent to the UV sterilizer;
wherein the silicon minerals column comprises silicon minerals, and the silicon minerals are prepared by stirring, mixing and sintering at a predetermined temperature, such that the silicon minerals are sintered into a crystalloid.

10. The method according to claim 9, wherein the predetermined temperature ranges from 0° C. to 60° C.

11. The method according to claim 9, wherein the silicon minerals form a mineral sphere having a diameter of 8 mm to 15 mm.

12. The method according to claim 9, wherein the activated carbon column, the at least one ion exchange resin column, the activated filter column, the Si minerals column, the UV sterilizer, and the magnetizer communicate with each other, and the drinking water is obtained by passing a tap water through the activated carbon column, the at least one ion exchange resin column, the activated filter column, the Si minerals column, the UV sterilizer, and the magnetizer.

13. The method according to claim 12, wherein the tap water sequentially passes through the activated carbon column, the at least one ion exchange resin column, the activated filter column, the Si minerals column, the UV sterilizer, and the magnetizer through a pressure gradient.

14. The method according to claim 13, wherein the activated carbon column, the activated filter column, and the magnetizer are in cylindrical forms, respectively.
